(19) United States
(12) Patent Application Publication
(10) Pub. No.: US 2024/0197904 A1
(43) Pub. Date: Jun. 20, 2024

(54) COMBINATION THERAPY FOR TREATING GLIOBLASTOMA

(71) Applicant: The Regents of the University of California, Oakland, CA (US)

(72) Inventors: David A. Nathanson, Los Angeles, CA (US); Timothy F. Cloughesy, Calabasas, CA (US); Elizabeth Fernandez, Los Angeles, CA (US)

(21) Appl. No.: 18/288,108

(22) PCT Filed: Apr. 25, 2022
(86) PCT No.: PCT/US22/26092

§ 371 Date: Oct. 24, 2023

Related U.S. Application Data
(60) Provisional application No. 63/179,769, filed on Apr. 26, 2021.

(51) Int. Cl. A61K 47/68; A61K 31/495; A61K 31/519; A61N 5/10; A61P 35/00

(52) U.S. Cl. CPC A61K 47/6849; A61K 31/495; A61K 31/519; A61K 47/6803; A61P 35/00; A61N 5/10

(57) ABSTRACT

The present disclosure relates to a method of treating a glioblastoma by conjointly administering to a subject an antibody-drug conjugate comprising a BCL-xL inhibitor and a second therapy such as an alkylating agent, irradiation, or an MCL-1 inhibitor.

### RELATED APPLICATIONS

The instant application claims priority to U.S. Provisional Patent Application No. 63/179,769, filed Apr. 26, 2021, the contents of which are hereby fully incorporated by reference herein.

### STATEMENT OF GOVERNMENT SUPPORT

This invention was made with government support under Grant Number CA213133, awarded by the National Institutes of Health. The government has certain rights in the invention.

### BACKGROUND

Glioblastoma (glioblastoma multiforme; GBM) accounts for the majority of primary malignant brain tumors in adults. Conventional therapies (e.g., temozolomide (TMZ), Irradiation (IR)) transiently halt tumor growth of glioblastoma (GBM) but fail to induce cell death through apoptosis. The inability to kill GBM tumor cells ultimately leads to disease progression and poor patient survival. The intrinsic apoptotic pathway is governed by the interactions of the BCL2 family of proteins which regulate the release of cytochrome C from the mitochondria. However, the precise molecular mechanisms that mediate apoptotic resistance in GBM remain enigmatic.

Molecular targeted therapies have revolutionized cancer treatment and paved the path for modern precision medicine. However, despite well-defined actionable genetic alterations, targeted drugs have failed in glioblastoma (GBM) patients. This is in large part due to insufficient CNS penetration of most targeted agents to levels necessary for tumor kill; potentially evoking robust adaptive mechanisms to drive therapeutic resistance. While drug combinations that inhibit both the primary lesion and the compensatory signaling pathway(s) are appealing, these combination therapy strategies have been hampered by enhanced toxicities leading to subthreshold dosing of each drug.

In view of the foregoing, there remains a clinical need for safe and effective combination or conjoint therapy for the treatment of glioblastoma and other cancers.

### SUMMARY OF THE INVENTION

In certain aspects, the present disclosure provides methods of treating glioblastomas, comprising conjointly administering to a subject in need thereof an antibody-drug conjugate comprising a BCL-xL inhibitor and a second therapy selected from an alkylating agent, irradiation, or an MCL-1 inhibitor.

### BRIEF DESCRIPTION OF THE DRAWINGS

FIG. 1 shows B7H3 expression in GBM autopsy samples.

FIG. 2A shows ABBV-155+IR specifically induces cell death in a B7H3 specific manner.

FIG. 2B shows ABBV-155+IR induces cell death in p53 WT GBM.

FIG. 3 shows the primed state of GBM predicts sensitivity to IR+ABBV-155 cell death.

FIG. 4 shows that ABBV-155+IR significantly extends survival in p53 WT model, SDX025.

FIGS. 5A & 5B show that intact p53 status alone may not predict the response to TMZ/IR in combination with BCL-Xli. FIG. 5A shows the apoptosis (Annexin V/PI+) of gliomaspheres (n=26) 5 days post IR (5gy) or TMZ (50 μM) and BCL-XLi (A1155463-0.5 μM) treatment normalized to either IR or TMZ alone (mean±s.d., n=2 independent replicates). The heat displays copy number alterations and mutations of p53, MDM2 and MDM4. MGMT status determined using MGMT methylation and expression, as described above. FIG. 5B shows the grouped analysis of all molecular biomarker positive samples (mut-p53/MDM2-4 WT Amp/MGMT positive) tumors vs all biomarker negative.

FIGS. 6A & B show that the functional assessment of the apoptotic ‘primed state’ alone may fully predict response to IR/TMZ and BCL-Xli. FIG. 6A shows basal BH3 profiling of gliomaspheres (n=26) preformed with a titration of the BIM peptide (0 μM, 0.01 μM, 0.03 μM, 0.1 μM, 0.3 μM, 1 μM, 3 μM, 10 μM). Data points in the dynamic range (0.03 μM-3 μM BIM) used to calculate area under the curve (AUC) to describe the primed state of these tumors. Diagram demonstrates examples of high and low primed cells. FIG. 6B shows grouped analysis of all functional biomarker positive samples (low primed) tumors vs all biomarker negative tumors (sub-divided into those that did and did not respond) both TMZ/IR and BCL-XLi cell death.

FIGS. 7A & 7B show that the integration of p53 signaling molecular status and the primed apoptotic state is superior to either molecular or functional biomarkers alone. FIG. 7A shows the correlations of BIM AUC with cell death induced by TMZ/IR and BCL-XLi, the grey band represents with 95% confidence interval and the line represents simple linear regression, used to calculate p-value and r-squared.

FIGS. 8A-8C show that B7H3 expression is higher in GBM tumors than the normal brain. FIG. 8A shows that immunohistochemistry staining for B7H3 (CD276) in GBM (n=33) and normal brain (n=5) microarray. Staining was quantified using the Precent Positive Pixels algorithm (ImageScope from Aperio), tumor presence was confirmed with a pathologist. FIG. 8B shows that B7H3 RNA expression in GBM tumors (TCGA n=157, patient tumors n=35, orthotopic xenografts n=37 and gliomaspheres n=26) compared to the normal brain (n=283). Data presented in counts per million, log 2 transformed. FIG. 8C shows immunoblot and quantification of B7H3 expression from normal brain (mean±s.d., n=5) and autopsy samples (mean±s.d., n=5).

FIG. 9 shows that Dynamic BH3 profiling reveals ABBV-155 creates a dependency on MCL-1 both in vitro and vivo. BH3 profiling 72 hours post ABBV-155 (1 μg/mL) treatment of gliomaspheres (n=6). Change in cytochrome c release calculated by determining the difference between vehicle and ABBV-155 treated gliomaspheres. To determine how the apoptotic dependencies are changing relative to basal MCL1 and BCL-XL blocks changes with individual peptides are subtracted the change in the HRK+MS1 condition. Finally, data were transformed by taking the z-score. In summary: Z-score across sample of (ΔHRK+MS1-(ΔHRK-100 μM or ΔMS1-10 μM or ΔABT-199-1 μM (Δ=TMZ/IR treated-Vehicle))). Heatmap displays these values. Workflow describing tumor purification of vehicle or ABBV-155 treated GBM orthotopic xenografts for Dynamic BH3 Profiling. Mouse whole brains were enzymatically digested, then red blood cells (RBC), myelin positive and mouse cells were removed. PDX39 (p53WT) was treated with one dose of ABBV-155 (10 mg/kg, i.p.). 10 days later purified tumor cells were used to preform ex-vivo dynamic BH3 profiling. Data indicates a change in cytochrome c release between ABBV-155 treated and vehicle untreated mice with MS1(MCL1−10 μM) (n=2 xenografts).

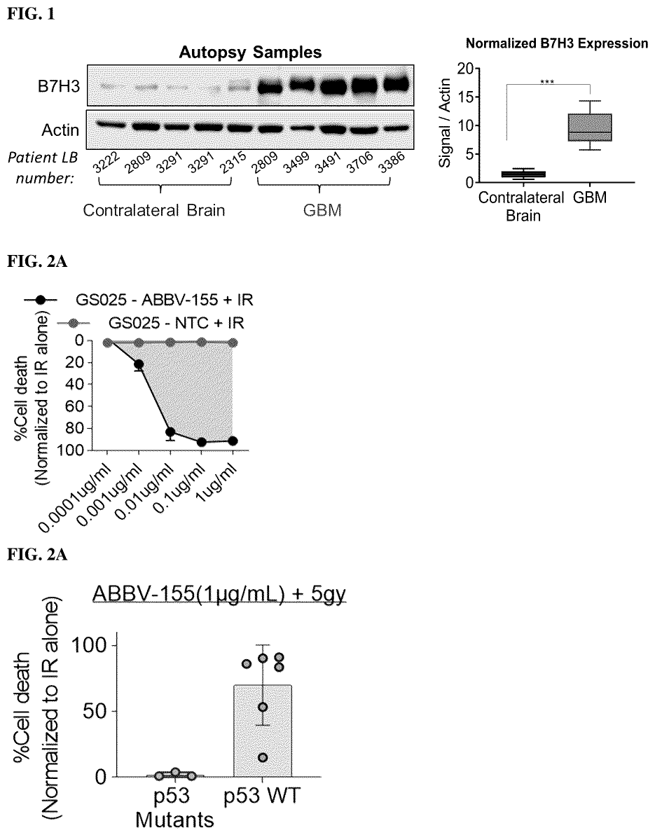

FIGS. 1A-C show that intact p53 signaling and a high primed state are important for sensitivity to ABBV-155 with TMZ/IR in gliomaspheres. FIG. 10A shows the Apoptosis (Annexin V/PI+) of p53 WT gliomaspheres, GS025, 5 days post IR (5gy) or TMZ (50 μM) and ABBV-155 titration or Non-Targeting Control (NTC). Concentration range: 0.0001 μg/mL, 0.001 μg/mL, 0.01 μg/mL, 0.1 μg/mL, 1 μg/mL. Data are normalized to either TMZ or IR alone (mean±s.d., n=2 independent replicates). FIG. 10B shows apoptosis (Annexin V/PI+) across gliomaspheres (n=7) 5 days post IR (5gy) or TMZ (50 μM) and ABBV-155 (0.0001 μg/mL, 0.001 μg/mL, 0.01 μg/mL, 0.1 μg/mL, 1 μg/mL) treatment. Data normalized to either IR or TMZ IR alone (mean±s.d., n=2 independent replicates). FIG. 10C shows the grouped analysis of all samples vs. p53 WT/MDM2-4 WT/MGMT negative and high primed tumors for both TMZ/IR and ABBV-155. Confidence interval on the right describes the difference between the means.

FIGS. 11A-D show that intact p53 signaling and a high primed state are important for sensitivity to ABBV-155 with IR in orthotopic xenografts. FIG. 11A shows representative mutations and copy number alterations in p53 and MDM2 for each PDX. FIG. 11B shows basal BH3 profiling of ex-vivo orthoptic xenograft tumors preformed with a titration of the BIM peptide (0 μM, 0.01 μM, 0.03 μM, 0.1 μM, 0.3 μM, 1 μM, 3 μM, 10 μM). Curve in the dynamic range (0.03 μM−3 μM BIM) used to calculate area under the curve (AUC) to describe the primed state of these tumors. Mean AUC used to categorize gliomaspheres as high or low primed. FIG. 11C shows the assessment of changes in PDX tumor growth with vehicle, IR, ABBV-155 or the IR/ABBV-155 combination. Following inoculation, the tumor burden of all mice was assessed twice weekly by secreted gaussia luciferase. Once tumors achieved exponential tumor growth, mice were randomized and treated with ABBV-155 (10 mg/kg, i.p. qw×3), IR (10 gy, qw×2), or the combination of ABBV-155 and IR. Fold change in tumor burden relative to size at enrollment displayed for all models (mean±s.d., n=8). Grouped comparisons made with a two-way ANOVA. FIG. 11D shows the percentage survival of orthotopic xenografts after the indicated treatments. Comparisons made using Log-rank (Mantel-Cox) test.

### DETAILED DESCRIPTION OF THE INVENTION

Gliomas are the most commonly occurring form of brain tumor, with glioblastoma multiforme (GBM) being most malignant form, causing 3-4% of all cancer-related deaths (Louis et al. (2007) Acta. Neuropathol. 114: 97-109.). The World Health Organization defines GBM as a grade IV cancer characterized as malignant, mitotically active, and predisposed to necrosis. GBM has a very poor prognosis with a 5-year survival rate of 4-5% with the median survival rate of GBM being 12.6 months (McLendon et al. (2003) Cancer. 98:1745-1748.). This can be attributed to unique treatment limitations such as a high average age of onset, tumor location, and poor current understandings of the tumor pathophysiology (Louis et al. (2007) Acta. Neuropathol. 114: 97-109). The current standard of care for GBM includes tumor resection with concurrent radiotherapy and chemotherapy and in recent years there have been few marked improvements that increase survival rates (Stewart, et al. (2002) Lancet. 359:1011-1018.).

The standard for GBM chemotherapy is temozolomide (TMZ), which is a brain-penetrant alkylating agent that methylates purines (A or G) in DNA and induces apoptosis (Stupp, et al. (2005) N. Engl. J. Med. 352:987-996). However, TMZ use has drawbacks in that significant risk arises from DNA damage in healthy cells and that GBM cells can rapidly develop resistance towards the drug (Carlsson, et al. (2014) EMBO. Mol. Med. 6: 1359-1370). As such, additional chemotherapy options are urgently required.

In embodiments of the methods herein, the subject has been diagnosed with glioblastoma multiforme. In some embodiments, the subject has been previously treated for glioblastoma with a prior treatment. In some such embodiments, the subject has been determined to be resistant to the prior treatment.

In certain embodiments of the current methods, the method further comprises administration of an additional therapy. In some embodiments, the additional therapy is radiation therapy, chemotherapy, targeted therapy, immunotherapy, or surgery. In some embodiments, the additional therapy comprises one or more therapies described herein.

### Malignant Gliomas and Glioblastomas—Types and Stages of Gliomas

Primary malignant brain tumors are tumors that start in the brain or spine are known collectively as gliomas. Gliomas are not a specific type of cancer but are a term used to describe tumors that originate in glial cells. Examples of primary malignant brain tumors include astrocytomas, pilocytic astrocytomas, pleomorphic xanthoastrocytomas, diffuse astrocytomas, anaplastic astrocytomas, GBMs, gangliogliomas, oligodendrogliomas, ependymomas. According to the WHO classification of brain tumors, astrocytomas have been categorized into four grades, determined by the underlying pathology. The characteristics that are used to classify gliomas include mitoses, cellular or nuclear atypia, and vascular proliferation and necrosis with pseudopalisading features. Malignant (or high-grade) gliomas include anaplastic glioma (WHO grade III) as well as glioblastoma multiforme (GBM; WHO grade IV). These are the most aggressive brain tumors with the worst prognosis.

In certain embodiments, the glioblastoma is p53 wild type glioblastoma.

GBMs is the most common, complex, treatment resistant, and deadliest type of brain cancer, accounting for 45% of all brain cancers, with nearly 11,000 men, women, and children diagnosed each year. GBM (also known as grade-4 astrocytoma and glioblastoma multiforme) are the most common types of malignant (cancerous) primary brain tumors. They are extremely aggressive for a number of reasons. First, glioblastoma cells multiply quickly, as they secrete substances that stimulate a rich blood supply. They also have an ability to invade and infiltrate long distances into the normal brain by sending microscopic tendrils of tumor alongside normal cells. Two types of glioblastomas are known. Primary GBM are the most common form; they grow quickly and often cause symptoms early. Secondary glioblastomas are less common, accounting for about 10 percent of all GBMs. They progress from low-grade diffuse astrocytoma or anaplastic astrocytoma, and are more often found in younger patients. Secondary GBM are preferentially located in the frontal lobe and carry a better prognosis.

GBM is usually treated by combined multi-modal treatment plan including surgical removal of the tumor, radiation and chemotherapy. In typical such conventional methods, as much tumor as possible is removed during surgery. The tumor's location in the brain often determines how much of it can be safely removed. After surgery, radiation and chemotherapy slow the growth of remaining tumor cells. The oral chemotherapy drug, temozolomide, is most often used for six weeks, and then monthly thereafter. Another drug, bevacizumab (known as Avastin®), is also used during treatment. This drug attacks the tumor's ability to recruit blood supply, often slowing or even stopping tumor growth.

Novel investigational treatments are also used and these may involve adding treatments to the standard therapy or replacing one part of the standard therapy with a different treatment that may work better. Some of these treatments include immunotherapy such as vaccine immunotherapies, or low-dose pulses of electricity to the area of the brain where the tumor exists and nano therapies involving spherical nucleic acids (SNAs) such as NU-0129. In some embodiments, the methods of the current disclosure are used in combination with one or more of the aforementioned therapies.

### Antibody-Drug Conjugates

### Antibody

In some embodiments, the antibody is specific for B7H3 (CD276). In certain embodiments, the antibody is 8h9, TPP-8382, TPP-8564, TPP-8567, enobilituzumab, omburtamab, or mirzotamab. In certain preferred embodiments, the antibody is mirzotamab. Additional antibodies specific for B7H3 are disclosed in US 20200199246, US 20170369585, WO 2017/214339, WO 2016/033225, WO 2004/001381, and WO 2020/103100, any of which could be utilized in the compositions and methods disclosed herein.

### Bcl-xL Inhibitors

In some embodiments, the Bcl-xL inhibitor is selected from, for example, WEHI 539, ABT-263, ABT-199, ABT-737, sabutoclax, AT101, TW-37, APG-1252, A-1155463, A-1331852 (clezutoclax), gambogic acid, any other Bcl-xL inhibitor, and combinations thereof. In certain embodiments, the BCL-xL inhibitor is A-1331852 or A-1155463. In certain preferred embodiments, the BCL-xL inhibitor is A-1331852.

### MCL-1 Inhibitors

In certain embodiments, the second therapy is an MCL-1 inhibitor. In some embodiments, the MCL-1 inhibitor is selected from, for example, AT-101, MIK665/S64315, AMG176, AMG-397, A-1210477, VU661013, AZD5991, S63845, any other MCL-1 inhibitor, and combinations thereof. In certain preferred embodiments, the MCL-1 inhibitor is S63845 or S64315.

In certain preferred embodiments, the ADC comprises an antibody specific for B7H3 (CD276) and a BCL-xL inhibitor and the MCL-1 inhibitor is S63845 or S64315. In certain particularly preferred embodiments, the ADC is ABBV-155 and the MCL-1 inhibitor is S63845 or S64315.

### Alkylating Agents

In certain embodiments, the second therapy is an alkylating agent. In further embodiments, the second therapy is an alkylating agent, such as temozolomide. In certain preferred embodiments, the ADC comprises an antibody specific for B7H3 (CD276) and a BCL-xL inhibitor and the second therapy is temozolomide. In certain particularly preferred embodiments, the ADC is ABBV-155 and the second therapy is temozolomide.

### Irradiation

In certain preferred embodiments, the second therapy is irradiation. In further preferred embodiments, the antibody is specific for B7H3 (CD276) and the second therapy is irradiation. In further preferred embodiments, the ADC is ABBV-155 and the second therapy is irradiation. In further embodiments, the method induces release of cytochrome C from glioblastoma cells. In yet further embodiments, the method induces apoptosis of glioblastoma cells.

### Methods of Treatment

The present disclosure provides methods of conjoint therapy that are useful in the treatment, prevention, or amelioration of GBM. In some embodiments, the methods treat, reduce or inhibit GBM in a subject. In some embodiments, the methods inhibit growth and/or proliferation of a GBM cell. In some embodiments, the methods improve the prognosis of GBM patients. In some embodiments, the methods reduce the risk of GBM. In some embodiments, the methods prime a GBM tumor cell to apoptosis. In some embodiments, the methods condition a GBM patient to treatment. In some embodiments, the methods reduce risk of ineffective therapy. In some embodiments, the methods ameliorate the symptoms of GBM. In some embodiments, the methods reduce the chances of tumor survival and/or recurrence. In some embodiments, the methods increase the vulnerability and/or sensitivity of tumor cells to therapy. The steps and embodiments discussed in this disclosure are contemplated as part of any of these methods. Moreover, compositions for use in any of these methods are also contemplated.

In various embodiments disclosed herein, the methods further comprise assessing the response of the patient to treatment.

In certain aspects, the present disclosure provides methods of treating glioblastoma in a subject, the method comprising conjointly administering to the subject a therapeutically effective amount of an antibody-drug conjugate targeting CD276 and comprising a BCL-xL inhibitor and a second therapy selected from an alkylating agent, irradiation, or (preferably) an MCL-1 inhibitor.

In certain embodiments of the methods herein, the subject has been diagnosed with glioblastoma multiforme. In some embodiments, the subject has been previously treated for glioblastoma with a prior treatment. In certain such embodiments, the subject has been determined to be resistant to the prior treatment. The methods of treatment disclosed herein may be useful even in the treatment of such resistant glioblastoma multiforme.

In certain embodiments of the current methods, the method further comprises administration of an additional therapy. In some embodiments, the additional therapy is radiation therapy, chemotherapy, targeted therapy, immunotherapy, surgery. In some embodiments, the additional therapy comprises one or more therapies described herein.

In certain embodiments, the methods herein comprise determining whether the glioblastoma is primed for apoptosis; and if the glioblastoma is primed for apoptosis; and if the glioblastoma is primed for apoptosis, administering the antibody-drug conjugate and the second therapy.

One or more compositions may be employed based on methods described herein, e.g., in the preparation of medicaments for treatments according to the methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

### Methods of Assessment

In some embodiments, a biological sample from a tumor is used. In other embodiments, a biological sample is blood or plasma. Evaluation of the sample may involve, though it need not involve, panning (enriching) for cancer cells, in vitro growth of cell line, or isolating the cancer cells.

The tumor biopsy may be but is not limited to GBMs. The methods of obtaining provided herein may include methods of biopsy such as needle aspiration, incisional biopsy, excisional biopsy, punch biopsy, or the like. The method of needle aspiration may further include fine needle aspiration, core needle biopsy, vacuum assisted biopsy, or large core biopsy. In some embodiments, multiple samples may be obtained by the methods herein to ensure a sufficient amount of biological material. In some cases, the fine needle aspirate sampling procedure may be guided by the use of an ultrasound, X-ray, or other imaging device.

In other embodiments the sample may be obtained from any of the tissues that include but are not limited to non-cancerous or cancerous tissue. General methods for obtaining biological samples are known in the art. Publications such as Ramzy, Ibrahim Clinical Cytopathology and Aspiration Biopsy 2001, which is herein incorporated by reference in its entirety, describes general methods for biopsy and cytological methods.

The biological sample may be blood or plasma, may be a heterogeneous or homogeneous population of cells. The biological sample may be obtained using any method known to the art that can provide a sample suitable for the analytical methods described herein.

The sample may be obtained by methods known in the art. In some cases, the sample may be obtained, stored, or transported using components of a kit of the present methods. In some cases, multiple samples, such as multiple cancerous samples may be obtained for diagnosis by the methods described herein. In other cases, multiple samples, such as one or more samples from one tissue type and one or more samples from another tissue may be obtained for diagnosis by the methods. Samples may be obtained at different times are stored and/or analyzed by different methods.

### Pharmaceutical Compositions

In certain aspects, the compositions or agents for use in the methods described herein, such as therapeutic agents or inhibitors, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices, local administration, and the like.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrante (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form.

Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide.

Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

### Dosage.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

In certain embodiments, pharmaceutical compositions may comprise, for example, at least about 0.1% of an active agent, such as therapeutic agents or diagnostic agents. In other embodiments, the active agent may comprise between about 2% to about 75% of the weight of the unit, or between about 25% to about 60%, for example, and any range derivable therein. In embodiments, the compositions are administered orally. In embodiments, the compositions are administered sublingually In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

The dosage of the pharmaceutical compositions and formulations depends on the type of formulation and varies according to the size and health of the subject. Various combination and dosages are contemplated and are within the scope of the current invention and within the scope of “effective dose”, “therapeutically effective dose”, “pharmaceutically acceptable” or “pharmacologically acceptable” compositions. Dosage of other therapeutics in accordance with the methods and compositions described herein are known in the medical community.

Upon formulation, solutions will be administered in a manner compatible with the dosage formulation and in such amount as is therapeutically or prophylactically effective. The formulations are easily administered in a variety of dosage forms, such as the sublingual, buccal, and transdermal formulations described above. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

### Combination Therapies

In certain embodiments, compounds of the invention may be used alone or conjointly administered with another type of therapeutic agent.

In certain embodiments, the methods of the current disclosure are used in combination with additional therapies such as chemotherapy, therapeutic agents, surgical removal of cancerous cells, radiation therapy, and combinations thereof. In some aspects, the treatment regimen excludes one or more of chemotherapy, therapeutic agents, surgical removal of cancerous cells and/or radiation therapy. When administered concurrently, the combination cancer therapies may be administered in a single formulation or in separate formulations, and if separately, then optionally, by different modes of administration.

In further embodiments a combination of therapeutic treatment agents is administered to cancer cells. The therapeutic agents may be administered serially (within minutes, hours, or days of each other) or in parallel; they also may be administered to the patient in a pre-mixed single composition.

Administration of the therapeutic compounds or agents to a patient will follow general protocols for the administration of such compounds, taking into account the toxicity, if any, of the therapy. It is expected that the treatment cycles would be repeated as necessary. It also is contemplated that various standard therapies, as well as surgical intervention, may be applied in combination with the described therapy.

Radiation therapy that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Alternative cancer therapy includes any cancer therapy other than surgery, chemotherapy and radiation therapy, such as immunotherapy, targeted therapy, gene therapy, or a combination thereof. Subjects identified with poor prognosis using the present methods may not have favorable response to conventional treatment(s) alone and may be prescribed or administered one or more alternative cancer therapy per se or in combination with one or more conventional treatments.

### Pharmaceutically Acceptable Salts

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds for laboratory use, or for subsequent conversion to a pharmaceutically acceptable acid addition salt.

The term “pharmaceutically acceptable basic addition salt” as used herein means any non-toxic organic or inorganic base addition salt of any acid compounds. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. In some embodiments it is contemplated that a numerical value discussed herein may be used with the term “about” or “approximately.”

The terms “attenuating”, “ameliorating,” “inhibiting,” or “reducing,” or any variation of these terms, when used in the claims and/or the specification includes any measurable decrease or complete inhibition to achieve a desired result.

The term “inhibitor” refers to a therapeutic agent that indirectly or directly inhibits the activity or expression of a protein, process (e.g. metabolic process), or biochemical pathway.

As used herein an “antagonist” describes a moiety that competitively binds to the receptor at the same site as an agonist, but does not activate the intracellular response initiated by the active form of the receptor and can thereby inhibit the intracellular responses by an agonist or partial agonist.

As used herein, in certain embodiments, “treating,” “treatment” or “therapy” is an approach for obtaining beneficial or desired clinical results. This includes: reduce the alleviation of symptoms, the reduction of inflammation, the inhibition of cancer cell growth, and/or the reduction of tumor size. In some embodiments, the term treatment refers to the inhibition or reduction of cancer cell proliferation in a subject having cancer. Furthermore, these terms are intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

In certain embodiments, the term “pharmaceutical formulation” or “pharmaceutical composition” is intended to mean a composition or a mixture of compositions comprising at least one active ingredient; including but not limited to salts, solvates, and hydrates of compounds described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The phrase “primed for apoptosis”, as used herein, refers to a cancer cell (e.g., a glioblastoma cell) which is more susceptible to apoptosis as compared to a typical cancer cell of the same type. The susceptibility of cancer cells (e.g., glioblastoma cell) to apoptosis can be measured by methods known to those of skill in the art. For example, the susceptibility of the cancer cell to apoptosis can be measured using BH3 profiling.

### EXAMPLES

The invention now being generally described, it will be more readily understood by reference to the following examples which are included merely for purposes of illustration of certain aspects and embodiments of the present invention, and are not intended to limit the invention.

### Example 1: Exemplary Treatment of GBM with BCLXL Inhibitor and IR

Antibody-drug conjugate ABBV-155 delivers a potent BCLXL inhibitor to GBM. ABBV-155 attaches A-1331852 (clezutoclax) to an antibody specific for B7H3 (CD276) (mirzotamab). B7H3 is highly expressed across GBM and minimally present in the normal brain (FIG. 1).

ABBV-155 is synergistically lethal with IR in p53 wild-type gliomaspheres in vitro. Apoptosis with Annexin V/PI staining was measured after treatment with ABBV-155. In p53 WT GBM, GS025, it was found that ABBV-155 in combination with IR synergistically induced cell death. IR combined with a non-targeted ADC had no effect (FIG. 2A). In total, it was found that the ABBV-155+IR has synergistically induced cell death in p53 WT GBMs (n=6) but had no effect in p53 mutants (n=3) (FIG. 2B). The p53 WT GBMs showed a differential sensitivity to IR+ABBV-155, similar to what we previously observed with the free BCLXLi, A1155463. It was found that the primed state of GBM, calculated using BIM area under the curve in the BH3 profiling assay, was predictive of sensitivity to IR+ABBV-155 (FIG. 3).

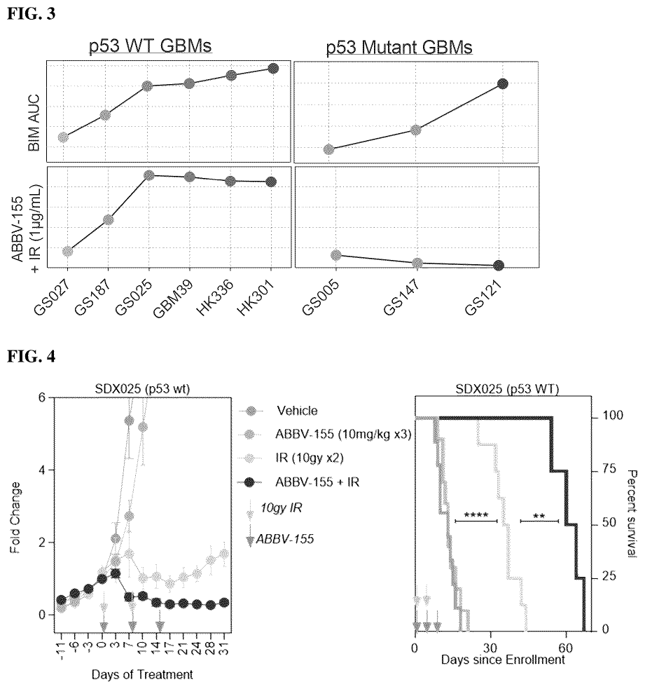

In vivo the p53 WT orthotopic xenograft, SDX025, showed no response to ABBV-155 (10 mg/kg×3) alone, both in tumor growth and in survival benefit. IR alone (10gy×2) produced a cytostatic effect, temporarily halting tumor growth and extending survival by 20 days. Only ABBv-155+IR promoted tumor burden decrease. This combination extended survival 40 days relative to vehicle and ABBV-155 alone (FIG. 4).

### Example 2: Further Exemplary Treatment of GBM with BCLXL Inhibitor and IR

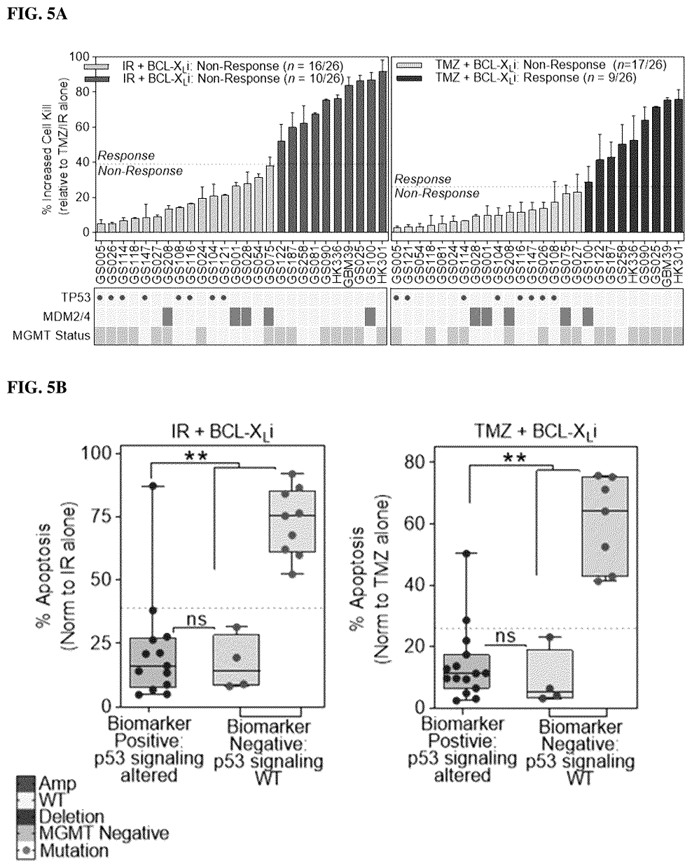

Mechanistic studies suggest that p53 mutation status may be used as a molecular biomarker to predict susceptibility to intrinsic apoptosis with either IR or TMZ combined with BCL-XL inhibition. To explore this observation, 26 gliomasphere lines were treated with either IR or TMZ plus BCL-XLi and assessed apoptosis relative to IR or TMZ alone. Intriguingly, while all p53 mutant GBMs (n=8) were insensitive to the combinations (determined by taking the mean of each treatment group), a substantial proportion (IR+BCL-XLi: n=8/18; TMZ+BCL-XLi: n=9/18) of p53 WT gliomaspheres were also non-responsive to the combination strategy (FIG. 5A). Thus, p53 mutational status alone cannot predict responses to targeting the GBM intrinsic apoptotic machinery with either IR or TMZ in combination with BCL-XL inhibition.

It was hypothesized that other molecular alterations within the p53 signaling pathway might affect the GBM tumor response to the IR or TMZ with BCL-XLi therapeutic combinations. For instance, MDM2 and MDM4 negatively regulate p53 signaling (Chene, 2003), and either MDM2 or MDM4 may be amplified in patients with wild type p53 GBM tumors (Tomczak et al., 2015). Indeed, both of the MDM2 amplified gliomaspheres and two of the three MDM4 amplified gliomaspheres, all of which are wild type p53, were insensitive to IR/TMZ+BCL-XLi (FIG. 5A).

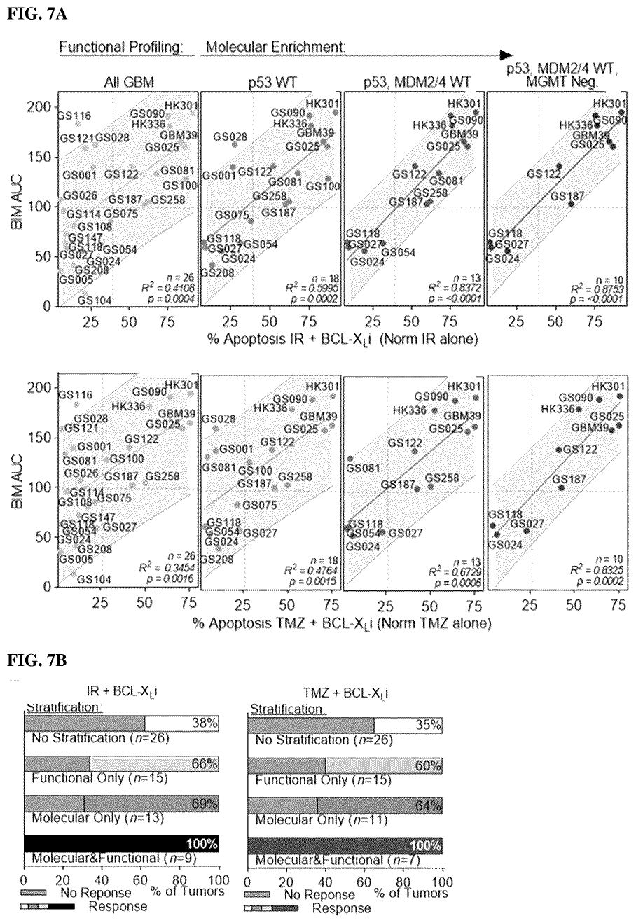

Activity of the DNA repair protein O-6-methylguanine-DNA methyltransferase (MGMT) repairs the DNA damage induced by TMZ treatment (Lalezari et al., 2013a). Consequently, GBMs with an unmethylated MGMT promoter and with high MGMT RNA expression (MGMT positive) are insensitive to TMZ treatment (Lalezari et al., 2013b). It was observed that most p53 WT gliomaspheres that were MGMT positive were non-responsive to TMZ and BCL-XLi (n=9/11) (FIG. 7A).

However, while incorporation of the status of both MDM2/MDM4 amplification and MGMT expression could further classify p53 WT GBMs insensitive to the combination strategy (FIG. 5A), there remained a considerable number of p53 WT gliomaspheres nonresponsive to treatment (IR+BCL-XLi: n=5/12; TMZ+BCL-XLi: n=3/9). Consequently, enrichment for GBMs with the compilation of molecular biomarkers (p53 WT, MDM2/MDM4 WT, and MGMT negative) was still unable to clearly distinguish between therapy responders and non-responders (FIG. 5B). Taken together, these findings suggest that our mechanistically defined molecular biomarkers cannot fully identify the GBMs sensitive to IR/TMZ+BCL-XL inhibition.

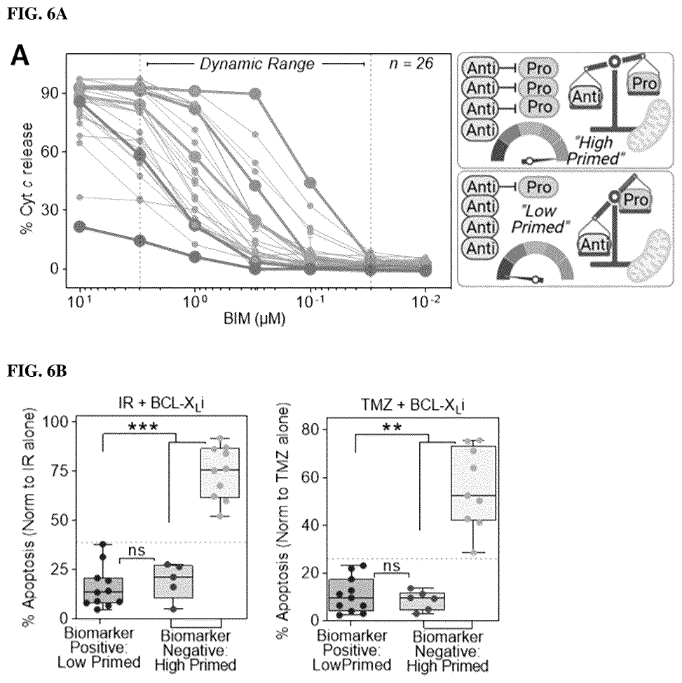

A functional assessment of a tumor's apoptotic potential (i.e., primed state) can strongly associate with therapy-induced intrinsic apoptosis (Certo et al., 2006). To explore whether apoptotic priming can identify GBMs responsive or non-responsive to IR/TMZ with BCL-XLi therapy, the apoptotic potentials of the panel of gliomaspheres (n=26) were measured by preforming BH3 profiling with increasing concentrations of the pro-apoptotic BIM BH3 peptide (equal affinity to all anti-apoptotic proteins (Kale et al., 2018b)) (FIG. 6A). The area under the curve of cytochrome c release was calcualted to create a metric to represent the primed state of each gliomasphere tested (BIM AUC).

Next, the differences in apoptotic potential among the gliomasphere cohort was assed to determine if it was possible to stratify responders and non-responders to IR/TMZ+BCL-XL treatment. Using receiver operative characteristic (ROC) curves to establish a binary classification of ‘high primed’ and ‘low primed’ gliomaspheres, it was observed that while all low primed gliomaspheres were insensitive to the IR/TMZ+BCL-XLi combination (FIG. 6B), there remained a considerable number of high primed gliomaspheres that did not respond to treatment (IR+BCL-XLi: n=5/15; TMZ+BCL-XLi: n=6/15) (FIG. 6B). Therefore, similar to what was observed with defined molecular biomarkers, functional BH3 profiling could not fully stratify GBMs responsive to this combination therapy approach.

To further explore the high primed GBMs that were insensitive to IR/TMZ+BCL-XLi, univariate linear regressions was performed to test the association between treatment-induced apoptosis and functional BH3 profiling (FIG. 7A). It was found that high primed gliomaspheres which demonstrated a low response to IR/TMZ+BCL-XLi also contained a molecular alteration in the p53 signaling pathway (e.g., GS121: mut-p53, GS028: MDM4 Amp). Therefore, it was hypothesized that enriching the population for gliomaspheres with intact p53 signaling (e.g., p53 WT, MDM2/4 WT) would enhance the association between apoptotic potential and drug sensitivity; adding these molecular features substantially strengthened the correlation between apoptotic priming and response to combined therapeutics (IR+BCL-XLi: R2=0.4108 4 0.8372; TMZ+BCL-XLi: R2=0.3454 4 0.6729) (FIG. 7A). Inclusion of MGMT status provided a substantial improvement to the TMZ+BCL-XLi combination (R2=0.8325) and but not with IR+BCL-XLi (R2=0.8753) (FIG. 7A). Consequently, stratifying GBMs by both p53 signaling status and apoptotic potential could now define 100% of the gliomaspheres that responded to IR/TMZ+BCL-XLi. In contrast, using p53 signaling status or BH3 functional profiling alone identified less than 70% of the gliomaspheres responsive to the therapeutic combination (FIG. 7B).

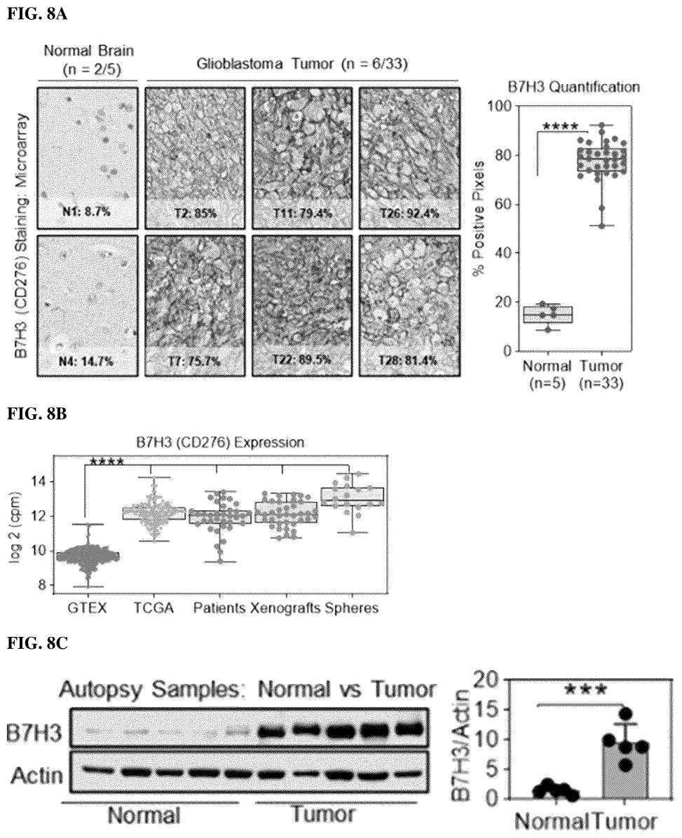

BCL-X_{L} is clinically difficult to target. BH3 mimetics that block BCL-XL (e.g., Navitoclax) have a poor therapeutic window in patients, due to on-target dose limiting toxicity in platelets, which depend on BCL-X_{L} for survival (Leverson et al., 2015). Mirzotamab Cletuzoclax (ABBV-155) is a first-in-class antibody-drug conjugate (ADC) that targets a cell surface antigen on B7-H3 (CD276) and includes a specific and potent BCL-X_{L} inhibitor warhead (Tolcher et al., 2021). B7-H3 is highly expressed in solid tumors, including glioblastoma, and has low protein levels in platelets and other normal tissues (Maachani et al., 2020; Nehama et al., 2019). Using a tissue microarray of GBM samples (n=33) and normal brain biopsies (n=5), high and diffuse B7H3 protein expression on GBM cells was observed, relative to normal brain specimens (FIG. 8A). B7H3 RNA expression in GTEX (normal brain) and TCGA GBM data sets was then assessed and compared the B7-H3 RNA levels to those of purified patient tumors, gliomaspheres and xenograft samples. Both the purified tumor specimens as well as GBMs in the preclinical models retained high levels of B7H3 relative to normal brain (FIG. 8B). These findings were confirmed by evaluating B7H3 protein expression in autopsy samples (FIG. 8C).

To determine if ABBV-155 could selectively abolish the BCL-XL block in gliomaspheres, dynamic BH3 profiling was performed across 6 gliomasphere lines (4 p53 WT, 2 mut-p53). ABBV-155 created a single dependency on MCL-1 to prevent MOMP in all gliomaspheres, confirming its ability to selectively remove the BCL-XL block regardless of genetic status (FIG. 8A).

It was then determined whether ABBV-155 could ablate the BCL-XL block in intracranial orthotopic GBM xenografts. GBM39 (p53 WT) patient-derived gliomaspheres were implanted into the brains of mice. Once tumors were established, mice were treated with one dose of ABBV-155 (10 mg/kg) or vehicle. Tumors were harvested 72 hours later and tumor cells purified and subjected to DBP. Consistent with the in vitro results, it was found that ABBV-155 caused a single dependency on MCL-1, indicating that the drug effectively ablated the BCL-XL block (FIG. 8B).

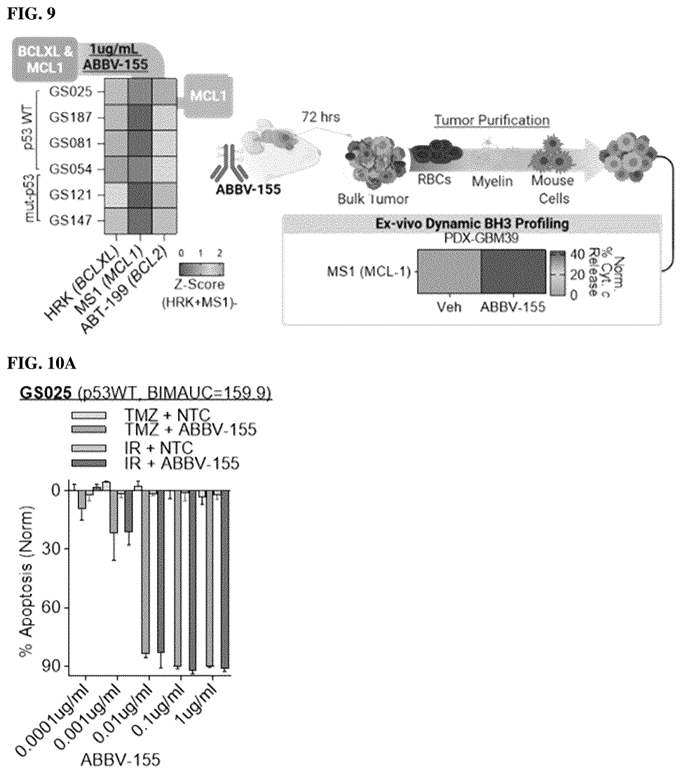

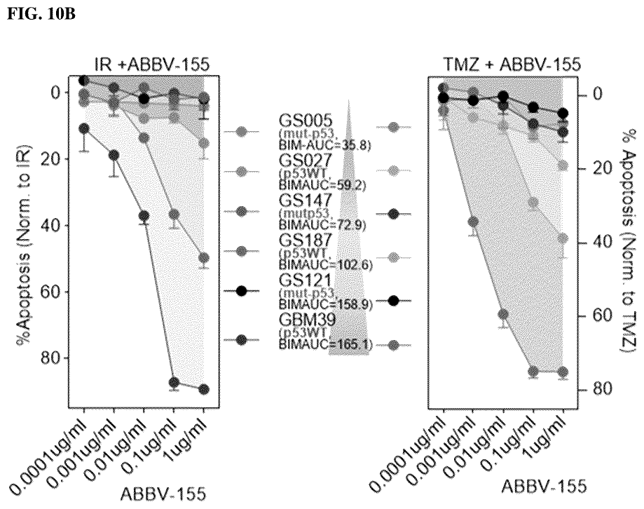

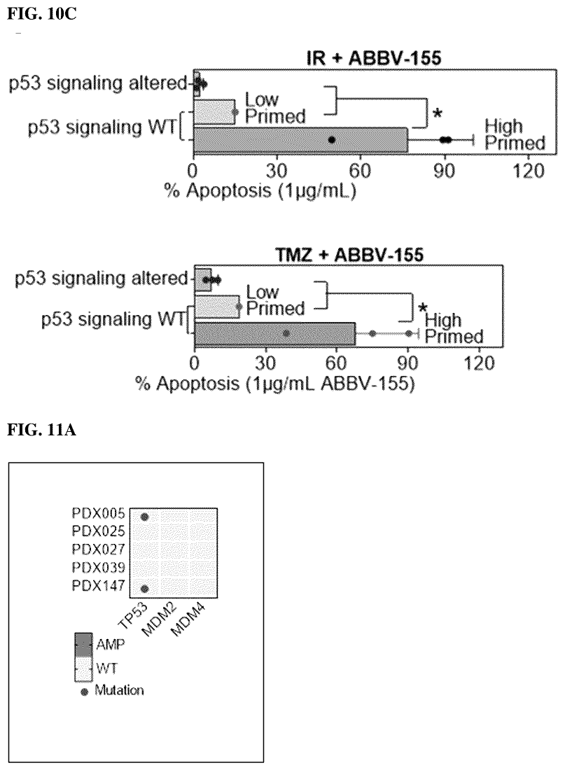

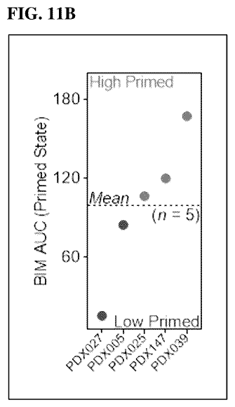

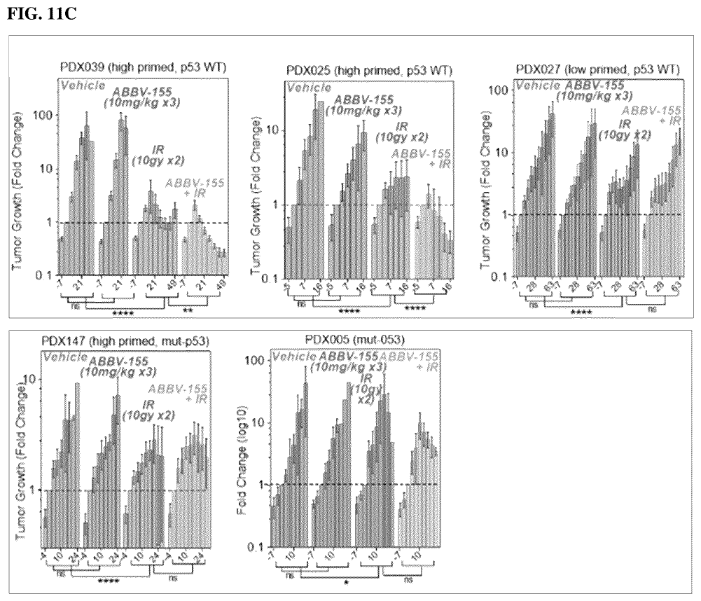

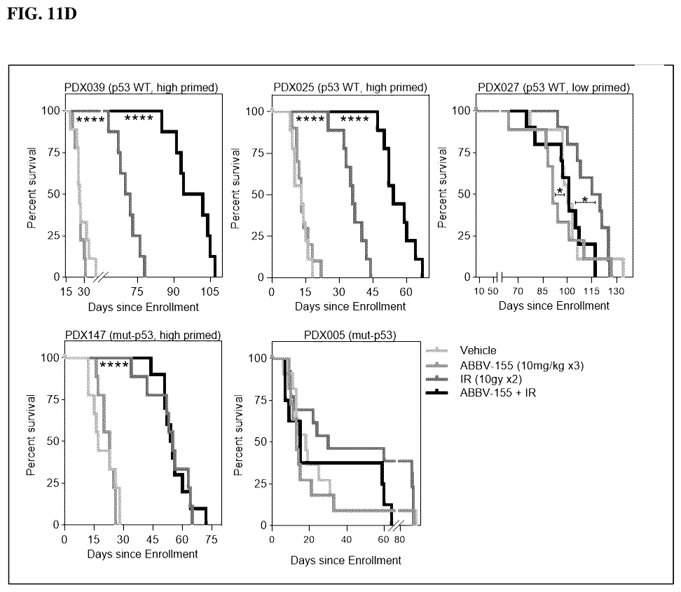

It was assessed if ABBV-155 is synergistically lethal with IR in p53 wild-type gliomaspheres in vitro. Apoptosis was measured with Annexin V/PI staining. In p53 WT GBM, GS025, it was found that ABBV-155 in combination with IR synergistically induced cell death. IR combined with a non-targeted ADC had no effect (FIG. 9A).

To summarize, ABBV-155+IR has synergistically induced cell death in p53 WT GBMs (n=6) but had no effect in p53 mutants (n=3) (FIG. 10B). The p53 WT GBMs showed a differential sensitivity to IR+ABBV-155, similar to what was previously observed with the free BCLXLi, A1155463. It was found that the primed state of GBM, calculated using BIM area under the curve in the BH3 profiling assay, was predictive of sensitivity to IR+ABBV-155 (FIG. 10C).

It was next explored whether ABBV-155 could sensitize GBMs to DNA damaging therapy in vivo. Cell culture data shown above demonstrate that enrichment for both molecular (p53 WT) and functional (high primed) features are required for sensitivity to either TMZ or IR in combination with ABBV-155. Next, it was determined if the predictive capabilities of this integrated molecular and functional approach would translate in vivo. To test this proposed predictive ability, next generation sequencing was performed (FIG. 11A) as well as ex vivo BH3 profiling (high primed state determined by mean, FIG. 11B) on a representative cohort (n=5) of patient derived orthotopic GBM xenografts. It was determined PDX025 and PDX039 were both p53 WT and high primed. Consequently, it was predicted these two GBM tumor xenografts would be sensitive to combination treatment. Conversely, PDX005 (mut-p53 and low primed), PDX027 (p53 WT and low primed) and PDX147 (mut-p53 and high primed) xenograft tumors lacked the molecular and/or functional characteristics proposed for therapeutic response to the combination therapy to either IR or TMZ in combination with ABBV-155. Thus, it was hypothesized these orthotopic xenografts would not respond to such combination therapies.

To test these predictions, a separate cohort of intracranial tumors was established from all 5 aforementioned PDXs. Following intracranial inoculation, mice were monitored for exponential tumor growth by secreted gaussia luciferase (Tannous, 2009a). Once aggressive growth was achieved, mice were randomized and treated with 10 mg/kg ABBV-155 IP qw for three weeks, 10gy IR qw for two weeks, or the combination of ABBV-155 and IR.

ABBV-155 did not impact tumor growth (in relation to vehicle treated mice) or survival in all PDX models tested. IR was capable of significantly inhibiting tumor growth—albeit largely cytostatic responses—and prolonging survival in both p53 WT and mut-p53 models, consistent with the observation that p53 status has no impact on the ability of IR to inhibit GBM growth (FIGS. 11C & 11D). In contrast, combined ABBV-155 and IR synergistically reduced tumor size and prolonged mouse survival; however, this combined therapeutic efficacy was confined to PDXs (PDX025 and PDX039) that are both p53 WT and high primed (FIGS. 11C 11). Taken together, these results demonstrate that the combination of DNA damaging therapy and ABBV-155 can have a synergistic anti-tumor impact in aggressive GBM PDX models that are both p53 WT and primed for apoptosis. The in vitro and in vivo findings demonstrate that integrated molecular and functional profiling can robustly predict responses to combined IR+ABBV-155 or TMZ+ABBV-155 therapeutic approaches to target GBM intrinsic apoptosis.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating a glioblastoma, comprising conjointly administering to a subject in need thereof an antibody-drug conjugate and a second therapy selected from an alkylating agent, irradiation, or an MCL-1 inhibitor, wherein the antibody-drug conjugate comprises a BCL-xL inhibitor.

2. The method of claim 1, wherein the BCL-xL inhibitor is selected from A-1331852, WEHI 539, ABT-263, ABT-199, ABT-737, sabutoclax, AT101, TW-37, APG-1252, A1155463, gambogic acid, and a combination thereof.

3. The method of claim 2, wherein the BCL-xL inhibitor is A-1155463 or A-1331852.

4. The method of claim 3, wherein the BCL-xL inhibitor is A-1331852.

5. The method of any one of claims 1-4, wherein the antibody-drug conjugate comprises an antibody specific for B7H3 (CD276).

6. The method of claim 5, wherein the antibody is enobilituzumab, omburtamab, or mirzotamab.

7. The method of claim 5, wherein the antibody-drug conjugate is ABBV-155.

8. The method of any one of claims 1-7, wherein the second therapy is an MCL-1 inhibitor.

9. The method of claim 8, wherein the MCL-1 inhibitor is selected from AT-101, MIK665/S64315, AMG176, AMG-397, A-1210477, VU661013, AZD5991, S63845 and a combination thereof.

10. The method of claim 9, wherein the MCL-1 inhibitor is S63845 or S64315.

11. The method of any one of claims 1-7, wherein the second therapy is selected from an alkylating agent and irradiation.

12. The method of claim 11, wherein the second therapy is an alkylating agent.

13. The method of claim 12, wherein the alkylating agent is temozolomide.

14. The method of claim 11, wherein the second therapy is irradiation.

15. The method of any one of claims 1-14, wherein the glioblastoma is p53 wild type glioblastoma.

16. The method of any one of claims 1-15, comprising determining whether the glioblastoma is primed for apoptosis; and if the glioblastoma is primed for apoptosis, administering the antibody-drug conjugate and the second therapy.

17. The method of any one of claims 1-15, wherein the method induces release of cytochrome C from glioblastoma cells.

18. The method of any one of claims 1-16, wherein the method induces apoptosis of glioblastoma cells.
